(19) United States
(12) Patent Application Publication
(10) Pub. No.: US 2021/0208074 A1
(43) Pub. Date: Jul. 8, 2021

(54) LIQUID TESTING APPARATUS AND METHOD

(71) Applicant: Validere Technologies Inc., Toronto (CA)

(72) Inventors: Ofer Vitner, Toronto (CA); Ian Burgess, Toronto (CA); Nouman Ahmad, Toronto (CA); John Nguyen, Toronto (CA)

(21) Appl. No.: 17/251,757

(22) PCT Filed: Jun. 11, 2019
(86) PCT No.: PCT/CA2019/050825

§ 371 Date: Dec. 11, 2020

Related U.S. Application Data
(60) Provisional application No. 62/683,623, filed on Jun. 11, 2018.
(60) Provisional application No. 62/683,625, filed on Jun. 11, 2018.

(51) Int. Cl. G01N 21/64; G01N 33/28; G01N 21/01; G01N 21/84; H04N 5/232; H04N 5/247; H04N 5/225

(52) U.S. Cl. CPC G01N 21/6456; G01N 33/2847; G01N 21/01; G01N 21/84; H04N 5/23206; G01N 2201/02; H04N 5/2256; H04N 5/23299; G01N 2021/8405; G01N 2201/062; H04N 5/247

(57) ABSTRACT

A testing apparatus includes a chamber. The chamber includes a holder configured to position a container. The chamber includes a first input device and a second input device, each partially outside the chamber and configured to: generate a first image of the container and generate a second image of the container. The chamber includes a first light source configured to illuminate the container for the first image. The chamber includes a second light source configured to illuminate the container for the second image.

### CROSS-REFERENCE TO RELATED APPLICATIONS

This application claims the benefit of U.S. Provisional Application No. 62/683,623, filed Jun. 11, 2018 and U.S. Provisional Application No. 62/683,625, filed Jun. 11, 2018, which are hereby incorporated by reference herein.

### BACKGROUND

Testing for liquid samples (e.g., hydrocarbons) can reveal the contents and quality of the samples. Machines and systems that perform liquid testing are prone to error from the placement of samples within the machine, from poor lighting, and from a lack of analysis capability.

### FIELD OF THE INVENTION

The present disclosure relates generally to liquid testing apparatus and methods, and more specifically, to liquid testing apparatus and methods using images.

### SUMMARY

In general, in one aspect, one or more embodiments relate to a testing apparatus that includes a chamber. The chamber includes a holder configured to position a container. The chamber includes a first input device and a second input device, each partially outside the chamber and configured to: generate a first image of the container and generate a second image of the container. The chamber includes a first light source configured to illuminate the container for the first image. The chamber includes a second light source configured to illuminate the container for the second image.

In general, in one aspect, one or more embodiments relate to a method for testing a hydrocarbon test sample. A first image of the hydrocarbon test sample is obtained via a first input device. The first input device is a primary camera configured to capture the first image while a plurality of light sources illuminates the hydrocarbon test sample. The first image is sent from the first input device to a control panel. The control panel labels a plurality of layers on the first image. A water cut of the hydrocarbon test sample is determined based on labeling of plurality of layers of the first image.

In general, in one aspect, one or more embodiments relate to a method for hydrocarbon testing comprising: inserting a container into a testing chamber; illuminating the testing chamber using a first light source; capturing a first image of the container with a first input device in proximity to the first light source illuminating the testing chamber; and sending the first image of the container from the input device to a computing device.

In general, in one aspect, one or more embodiments relate to a testing apparatus that includes a chamber. The chamber includes a holder configured to position a container. The chamber includes a first input device, partially outside the chamber and configured to generate a first image of the container. The chamber includes a first light source configured to illuminate the container for the first image.

### BRIEF DESCRIPTION OF DRAWINGS

The present embodiments are illustrated by way of example and are not intended to be limited by the figures of the accompanying drawings.

FIGS. 1A and 1B show an example of a testing apparatus in isometric and side view, respectively, in accordance with one or more embodiments.

FIGS. 2A and 2B show an example of a testing apparatus in isometric and side view, respectively, in accordance with one or more embodiments.

FIGS. 3A and 3B show an example of a testing apparatus in isometric view in accordance with one or more embodiments.

FIG. 4 shows a side view is shown of a testing apparatus similar to FIGS. 3A and 3B in accordance with one or more embodiments.

FIG. 5 shows top view example of a testing apparatus similar to FIGS. 3A and 3B in accordance with one or more embodiments.

FIG. 6 shows an example of a testing apparatus in an isometric view in accordance with one or more embodiments.

FIG. 7 shows a top view of a testing apparatus similar to FIG. 6 in accordance with one or more embodiments.

FIG. 8 shows an exploded view of the testing apparatus from FIG. 6 in accordance with one or more embodiments.

FIGS. 9A and 9B show an isometric and top view, respectively, of one side of the testing apparatus in accordance with one or more embodiments.

FIG. 10 shows a section view of FIG. 6 taken from the inside of the testing apparatus in accordance with one or more embodiments.

FIG. 11 shows an example of a top view of a testing apparatus m accordance with one or more embodiments.

FIG. 12 shows an example of a top view of a testing apparatus m accordance with one or more embodiments.

FIG. 13 shows a flow diagram of a system for testing in accordance with one or more embodiments.

FIG. 14 shows a flowchart describing a method for capturing an image in accordance with one or more embodiments.

### DETAILED DESCRIPTION

The following detailed description is merely exemplary in nature, and is not intended to limit the disclosed technology or the application and uses of the disclosed technology. Furthermore, there is no intention to be bound by any expressed or implied theory presented in the preceding technical field, background, or the following detailed description.

In the following detailed description of embodiments, numerous specific details are set forth in order to provide a more thorough understanding of the disclosed technology. However, it will be apparent to one of ordinary skill in the art that the disclosed technology may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

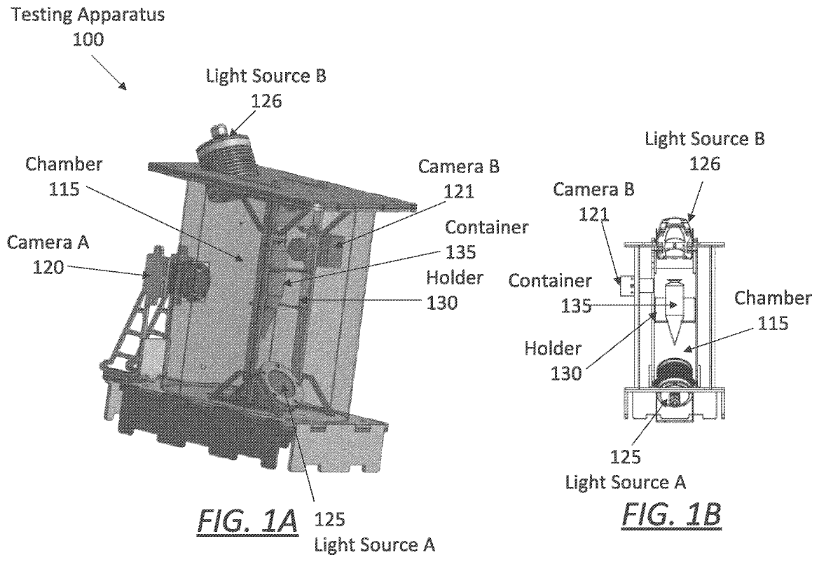

Turning now to the figures, FIGS. 1A and 1B show different views of a testing apparatus (100) in accordance with one or more embodiments of the invention. FIG. 1A shows an isometric view of an embodiment that includes one or more chambers (e.g., chamber (115)), which are areas where the various components are located. In one or more embodiments, the various components include the input devices (e.g., camera A (120), camera B (121), etc.), the container (135) that is commonly shaped as a tube that is suspended in the chamber (115) by a holder (130), and various different light sources (e.g., light source A (125), light source B (126), etc.). In one or more embodiments, one or more chambers (e.g., chamber (115)), are bounded by four walls encompassing an enclosed space. The arrangement of the components within the chamber depends heavily on the manner and type of testing performed by the testing apparatus (100).

FIG. 1B shows a side view from the position of camera A of an embodiment of the invention that includes a chamber (115) within which each of the component are at least partially contained. In one or more embodiments, the various components include the input devices (e.g., camera B (121), etc.), the container (135) that is commonly shaped as a tube that is suspended in the chamber (115) by a holder (130), and a light source (e.g., light source A (125), etc.).

Each of the components are described below.

The input devices, such as camera A (120) and camera B (121) may be integrated camera(s) as part of a tablet-based control panel (not shown), and/or industrial camera(s) depending on the particular need for the testing apparatus (100). In one or more embodiments, camera A (120) and camera B (121) may be placed within the testing apparatus (100) at a position 90 degrees with respect to each other, in order to gather more information from the different angle. Camera A (120) and camera B (121) may also be positioned at any angle with respect to each other. Camera A (120) and camera B (121) may also be at different heights within the chamber (115) to focus on different areas of interest.

In one or more embodiments, the container (135) (or tube) may be either long/short, per ASTM D4007 and/or D0097. In one or more embodiments, the position of the calibrated volume marks found on the container (135) are in front of a primary camera (e.g., camera A (120)). The holder (130) within the chamber (115) may be able to adjust to host either of the long/short containers. In one or more embodiments of the invention, the testing apparatus (100) is capable of acquiring an image of a pair of containers at a time, so two holders may be necessary.

In one or more embodiments, various different light sources (e.g., light source A (125), light source B (126), etc.) may be of any type now known or developed in the future that are capable of facilitating the ability to learn and analyze as much as possible from every bit of data from one or more images pictures coming from different input devices. The intensity, color, shape, size, angle, and the ability to control light sources separately, provides the flexibility to superimpose or manipulate the images obtained by using all/part of the light sources in accordance with one or more embodiments. Because the color of the testing sample (e.g., a hydrocarbon testing sample) varies form crystal clear to “coal black”, the light source(s) reduce reflections using post processing manipulations.

In one or more embodiments, the light sources may be ultraviolet (UV) light. It is important to understand that UV light provides another layer of analytics. To a degree, using UV light allows for detecting other materials which may be defined as soluble, yet others are not. For example, the UV light source may determine additional layers such as but not limited to: trapped water in tight emulsions, paraffin waxes, asphaltenes, drilling fluids, and other contaminants or additives. The image captured using the UV light source may show an approximation of trapped water due to the differences in fluorescence of water and oil. UV image acquisition and analysis uses differences in fluorescence in all of the constituents of the testing sample relative to their appearance in visible light. Simultaneous analysis of visible light illuminated images and UV illuminated images improve constituent classification capabilities. The UV light may be positioned in any placement around the chamber (115) where a light source is feasible.

In one or more embodiments, the light sources may have different angles, heights, shapes, and types. In particular, the type of light sources may include LEDs, UV in a ‘ring’ like shape together with ‘white’ LEDs on the same ring PCB, a long bar with an array of LEDs illuminating a cylindrical chamber in order to minimize reflection and provide homogeneous light around the container without directly ‘hitting’ the container, as well as short arrays of LEDs, around the tube in different heights, illuminating the container directly, with different angles.

In one or more embodiments, the chamber (115) is a flat shape or may be a round, cylindrical shape. The round, cylindrical shape behind the container (135) improves the illumination of the container (135) when using indirect light by providing light arrays to reflect from that curved wall backwards to the ‘back’ side of the container (135). In one or more embodiments, the color painted in the chamber (115) is also of an importance, to assure good analytics of the testing sample, regardless of the sample color, under different light conditions.

One or more embodiments of the testing apparatus (100) provides one or more the following advantages or benefits. The testing apparatus (100) is capable of recording sample data for use during future disputes. The testing apparatus (100) exports the data (e.g., image(s), user data, materials added to the sample, type of sample, etc.) via WiFi/cellular communication to a secured database. The testing apparatus (100) may work in online as well as offline modes. The testing apparatus (100) may be introduced to harsh conditions. The testing apparatus (100) may be used both indoors and outdoors. The testing apparatus (100) may be portable and mounted on a vehicle per need. The testing apparatus (100) may be both corded or cordless (using a battery). The service(s) associate with the testing apparatus (100) provides value—a number in which the tester knows the quantity of different materials in that sample. The service(s) associate with the testing apparatus (100) may provide a set of analytical tools related to the suppliers of customers, consistent quality, integrity, internal quality procedures and processes, etc.

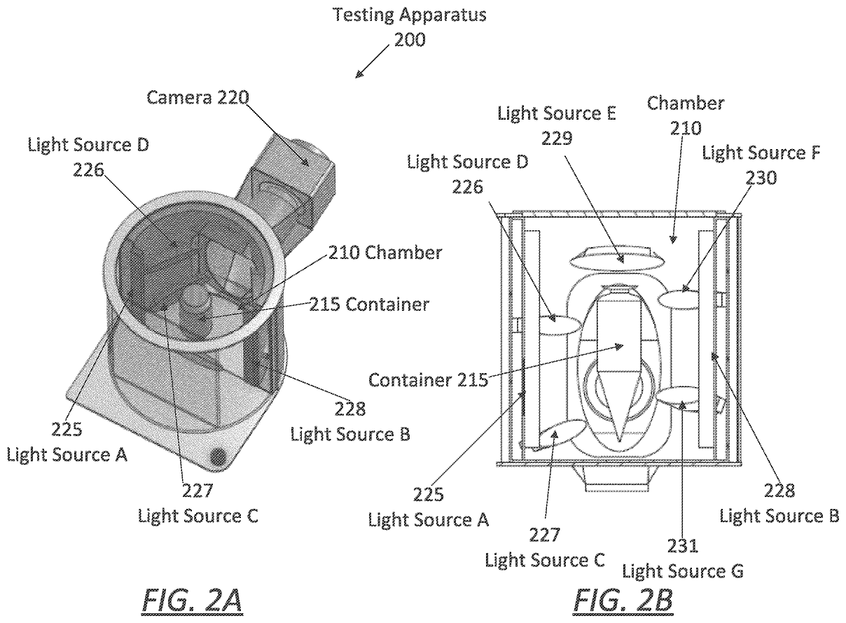

FIGS. 2A and 2B show a testing apparatus (200) in accordance with one or more embodiments of the invention. The testing apparatus (200) show a different embodiment with many of the same features shown and described in relation to FIGS. 1A and 1B. In particular FIGS. 2A and 2B show a camera (220), multiple lights sources (e.g., light source A (225), light source B (228), light source C (227), light source D (226), light source E (229), light source F (230), light source G (230)), a container (215), and a chamber (210).

Each of the components are described below.

The camera (220) may be a similar camera as described in FIG. 1. In one or more embodiments, the camera (220) is located on one end of a shaft extending from the chamber (210). The camera (220) is positioned in a manner to focus on the container (215) located in the center of the chamber (210). The container (215) contains the hydrocarbon test sample and may be labeled with volume marks to show the volume of the container (215).

In one or more embodiments, light source A (225) and light source B (228) are vertical bar lights oriented in a vertical direction along opposite walls of the chamber (210). Light source A (225) and light source B (228) may be similar to the light sources described in FIG. 1. One of ordinary skill in the art will appreciate that the placement of light source A (225) and light source B (228) in FIG. 2A differs from the placement shown in FIG. 1, however, light sources are able to illuminate the container (215) in both figures. Additionally, FIG. 2A adds a light source C (227) and light source D (226). Both light source C and light source D may be bar lights placed in a horizontal direction and perpendicular to light source A (225). In one or more embodiments, light source C (227) may be placed directly below light source D (226) at a predetermined height or any height. In one or more embodiments, the placement oflight source A (225), light source B (228), light source C (227), and light source D (225) may be such that the light sources illuminate a wall within the chamber (210) that reflects onto the container (215).

FIG. 2B shows a cross section view of FIG. 2A. In addition to light source A (225), light source B (228), light source C (227), and light source D (226), FIG. 2B shows the placement of a light source E (229), a light source F (230), and a light source G (231) located within the chamber (210). In one or more embodiments, the placement of light source E (229) is located above the shaft opening into the chamber (210). Light source E (229) may be placed at an angle that allows for the light source to illuminate directly on the container (215). In one or more embodiments, light source E (229) may be placed at a different angle to allow the light source to illuminate the container (215) indirectly (i.e., reflection off a wall). In one or more embodiments, light source F (230) and light source G (231) may be light bars placed in a horizontal direction and perpendicular to light source B (228). In one or more embodiments, light source G (231) may be placed directly below light source F (230) at a predetermined height or at any height. One of ordinary skill in the art will appreciate that the placement of the light sources (e.g., light source A (225), light source B (228), light source C (227), light source D (226), light source E (229), light source F (230), light source G (230)) is not limited to the embodiment discussed, and that the light sources may be positioned in any number of ways around the chamber (210) to illuminate the container (215).

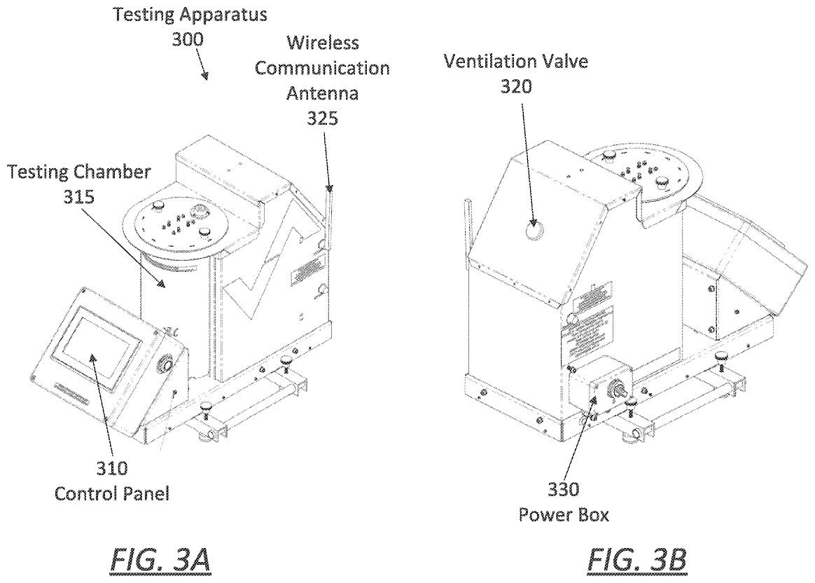

FIGS. 3A and 3B show an example of a testing apparatus (300) in isometric view. As shown in FIG. 3A, one side of the testing apparatus includes of a control panel (310) a wireless communication antenna (325), and a testing chamber (315). FIG. 3B shows the opposite side of FIG. 3A, which includes a ventilation valve (320) and a power box (330). In one or more embodiments, the testing apparatus (300) may be used to test hydrocarbons.

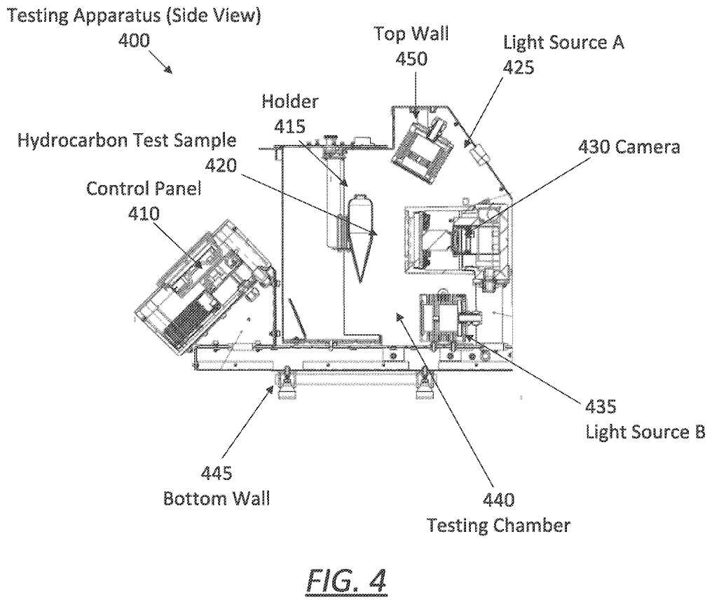

In one or more embodiments, a control panel (310) is located on the outer portion of the testing apparatus (300). The control panel (310) may be located separate from the testing chamber (315). In one or more embodiments, the control panel (310) is aligned and part of the testing chamber (315). The control panel (310) may be angled to allow a user clear visibility of the display screen. For example, prior tests may be carried out to determine that the ideal viewing angle for the control panel (310) is 45 degrees. The control panel (310) is discussed below and in relation to FIG. 4.

The testing chamber (315), in accordance with one or more embodiments, is located in the central portion of the testing apparatus (300). In one or more embodiments, the testing chamber (315) is cylindrical. The testing chamber (315) may be made of the same material as the testing apparatus (300) such as stainless steel, aluminum, copper-aluminum alloy, non-sparking metal, carbon steel, class 1 div 2 explosion proof material, iron, carbon fiber, plastic, or any other type of material. The inside of the testing chamber (315) may be a different color than the rest of the testing apparatus (300). For example, the inside of the testing chamber (315) may be painted red to allow for better viewing and operating conditions of the camera (not shown) and better illumination of the multitude of light sources (not shown). In one or more embodiments, the outer wall of the testing chamber (315) may rotate to allow access inside.

In one or more embodiments, the wireless communication antenna (325) is located on the outer portion of the testing apparatus (300). The wireless communication antenna (325) may contain capability to send data over a wireless network to another computing device (not shown). The wireless communication antenna (325) may utilize any type of wireless network (e.g., WiFi, Bluetooth, cellular network). For example, a user may wish to send data stored on the control panel (310) to another computing device. The wireless communication antenna (325) allows the user to send the data over a wireless network, such as WiFi.

In one or more testing apparatus embodiments, a ventilation valve (320) is positioned on the outer portion of the (300) as shown in FIG. 3B. The ventilation valve (320) may be opened and closed to allow air ventilation into the testing chamber or allow for gas to escape the testing chamber (315). For example, a hydrocarbon test sample may be placed inside the testing chamber (315) and the ventilation valve (320) is opened to allow for flammable gas to escape and not build up inside the testing chamber (315). The ventilation valve (320) may be made of the same material as the testing apparatus (300) such as stainless steel, aluminum, copper-aluminum alloy, non-sparking metal, carbon steel, class 1 div 2 explosion proof material, iron, carbon fiber, plastic, or any other type of material.

In one or more embodiments, a power box (330) is located on the outside of the testing apparatus (300). In one or more embodiments, the power box (330) may be located inside the testing apparatus (300). The power box (330) may include of an on/off switch, button, or any other type of mechanism to allow a user to turn the power on and off The power box (330) may be encased in the same material as the testing apparatus (300) such as stainless steel, aluminum, copper-aluminum alloy, non-sparking metal, carbon steel, class 1 div 2 explosion proof material, iron, carbon fiber, plastic, or any other type of material.

Turning to FIG. 4, a side section view is shown of a testing apparatus (400) similar to FIGS. 3A and 3B. As shown in FIG. 4, the testing apparatus includes of a control panel (410) located on the outside and a testing chamber (440), which is separate from the control panel (410). The testing chamber (440) is defined by a top wall (450) and a bottom wall (445). A first light source (e.g., light source A (425)) is located on the top wall (450) inside of the testing chamber (440) while a second light source (e.g., light source B (435)) is located on the bottom wall (450) inside of the testing chamber (440). A holder (415) extends from the top wall (450) towards the bottom wall (445). A hydrocarbon test sample (420) is positioned in the holder (415) and a camera (430) is located opposite of the hydrocarbon test sample (420).

In one or more embodiments, the control panel (410) extends from the bottom wall (445) outside of the testing apparatus. The control panel (410) is positioned in such a manner that allows a user to view and interact with the control panel (410) while the testing apparatus (400) rests in a horizontal position. In one or more embodiments, the control panel (410) allows the user to control the camera (430) as well as the first light source (425) and the second light source (435). For example, the user may use the control panel (410) to use the camera (430) to zoom in and out, focus the camera (430) adjust the flash settings of the first light source (425) and the second light source (435), and provide input to capture an image using the camera (430) while the first light source (425) and second light source (435) illuminate the testing chamber. In one or more embodiments, the control panel (410) includes of a GPS device that allows the multitude of images to be tagged with a geographic location. For example, when an image is captured by the camera (430), the image is sent to the control panel via a network (e.g., wireless network, Bluetooth, cellular network), a connection cable (e.g., USB cable, ethernet cable, VGA cable). Or a portable storage device (e.g., USB drive, portable hard drive).

In one or more embodiments, the control panel (410) allows the user to view the multitude of images captured by the camera (430). The user may analyze the multitude of images on the control panel (410), or the control panel may analyze the multitude of images using image processing.

The control panel (410), in accordance with one or more embodiments, may identify errors in the multitude of images through the use of image processing. For example, the control panel (410) may run an algorithm to check that the layers of an image are labeled correctly. If a label is found to be incorrect, the control panel (410) may notify a user on the display screen that there is an error with the image. In one or more embodiments, the control panel (410) may identify errors in the image based on data collected from online analyzing tools. For example, the control panel (410) may detect that an image tagged with a certain geographic location has data that conflicts with data from an online analyzing tool that has a similar geographic location. An alert is sent to the display screen of the control panel (410) and a user may verify the error. In one or more embodiments, the alert is sent to a mobile device of the user or a separate computing device.

In one or more embodiments, a holder (415) extends from the top wall (450) to the center of the testing chamber (440). The holder (415) is attached to the top wall (450) via a fastening device (e.g., a screw, nut and bolt, pin). The holder (415) may be made out of the same material as the testing apparatus (400) or a different material entirely. For example, the holder (415) may be made out of stainless steel, aluminum, copper-aluminum alloy, non-sparking metal, carbon steel, class 1 div 2 explosion proof material, iron, carbon fiber, plastic, or any other type of material. The holder (415) may be of any length that allows for the hydrocarbon test sample (420) to rest opposite of the camera (430).

The hydrocarbon test sample (420), in accordance with one or more embodiments, is a container that includes of a mixture of liquids, gases, and solids. The hydrocarbon test sample (420) may be of any shape suitable for containing the mixture. For example, the hydrocarbon test sample (420) may be a test tube, a cylindrical tube, a flask, a beaker, or any other type of container holds a liquid. For example, the container may be a tube that is manufactured in accordance with ASTM D4007 or ASTM D0097. The hydrocarbon test sample (420) may be made of glass, plastic, acrylic glass, or any other type of material which allows for the user, camera (430), and control panel (410) to see the liquid, solid, and gas phases. In one or more embodiments, the hydrocarbon test sample (420) is labeled with volume marks to represent the volume of the container. The volume marks are located in a manner to be visible by the set of cameras. The volume may be indicated in liters, milliliters, cm³, fluid ounces, or any other type of measure for volume.

In one or more embodiments, the testing apparatus (400) includes of a first light source (425) and a second light source (435). The first light source (425) may be located on the top wall (450) while the second light source (435) may be located on the bottom wall (445). Both the first light source (425) and second light source (435) may be attached to the testing apparatus (400) via a fastening device (e.g., a screw, nut and bolt, pin) or a support mechanism. For example, the first light source (425) may be attached to the top wall (450) by a support rod that extends down from the top wall (450). The support rod may be fastened to both the top wall (450) and the first light source (425) by screws. However, the second light source (435) may be fastened to the bottom wall (445) with screws only. The first light source (425) and second light source (435) may be a LED strip, a LED bulb, or any other type of light source that illuminates the testing chamber. In one or more embodiments, the first light source (425) and the second light source (435) may be encased in the same material as the testing apparatus (400), such as stainless steel, aluminum, copper-aluminum alloy, non-sparking metal, carbon steel, class 1 div 2 explosion proof material, iron, carbon fiber, plastic, or any other type of material. For example, the first light source (425) and second light source (435) may both be encased in a class 1 div 2 explosion proof material.

In one or more embodiments, the camera (430) is located on a wall opposite the hydrocarbon test sample (420) and testing chamber. The camera (430) may be any type of input device capable of capturing a multitude of images. In one or more embodiments, the camera (430) is attached to the testing apparatus (400) via a mounting device. The mounting device may be attached to both the camera (430) and testing apparatus via a fastening device (e.g., a screw, nut and bolt, pin). In one or more embodiments, the camera (430) may be encased in the same material as the testing apparatus (400), such as stainless steel, aluminum, copper-aluminum alloy, non-sparking metal, carbon steel, class 1 div 2 explosion proof material, iron, carbon fiber, plastic, or any other type of material. For example, the first camera (430) may both be encased in a class 1 div 2 explosion proof material.

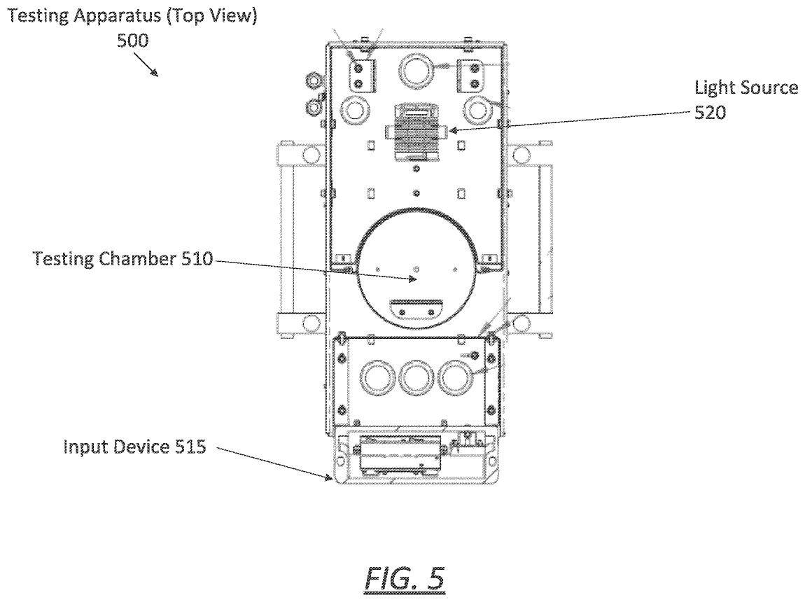

FIG. 5 shows top view example of a testing apparatus (500) similar to FIGS. 3A and 3B. As shown, the testing apparatus (500) includes of a light source (520) located inside the testing apparatus (500). The light source (520) is facing towards a testing chamber (510). A control panel (515) is located on the outer portion of the testing apparatus (500).

In one or more embodiments of the invention, the testing apparatus (500) includes of a testing chamber (510) that is centrally located. The testing chamber (510) may be shaped like a cylinder to allow for better illumination of the testing chamber (510) from the light sources (520). The testing chamber (510) may be made of the same materials as the testing apparatus (500). The testing chamber (510) may be made of stainless steel, aluminum, copper-aluminum alloy, non-sparking metal, carbon steel, class 1 div 2 explosion proof material, iron, or any other type of material. For example, the testing chamber (510) may be made out of a stainless steel rated for a class 1 div 2 hazardous area classification to ensure that the testing chamber (510) operates safely in conditions containing hazardous vapors, gases, or other flammable substances that could result in an explosion.

In one or more embodiments, a light source (520) is located on the top wall of the testing apparatus (500) inside the testing apparatus. As previously discussed, the light source (520) may be angled towards the hydrocarbon test sample. In one or more embodiments, the light source (520) is a LED light.

In one or more embodiments, a control panel (515) is located on the outside of the testing apparatus (500). The control panel (515) allows for a user to interact with the testing apparatus (500) by viewing a multitude of images captured in the testing chamber (510). In one or more embodiments, the control panel (515) allows the user to operate the camera (not shown) and light sources (520) located inside the testing apparatus (500).

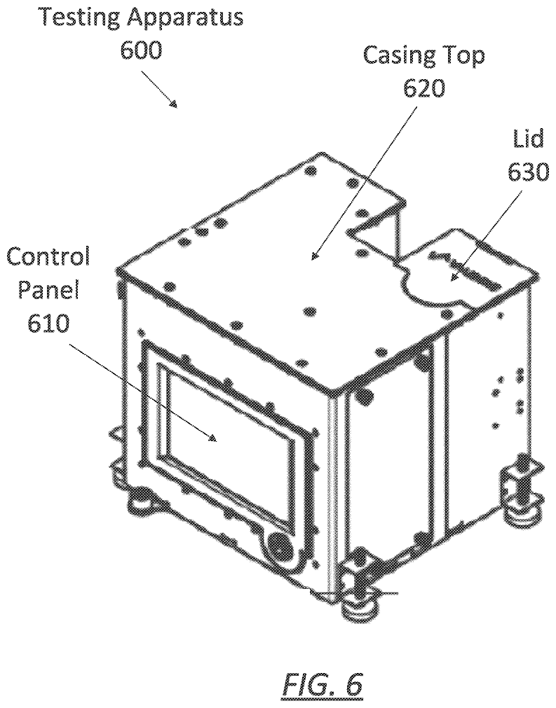

FIG. 6 shows an example of a testing apparatus (600) in an isometric view. As shown in FIG. 6, the outside of the testing apparatus (600) contains a control panel (610) located on one wall and a lid (620) located on the top portion (abutting the casing top (620)) of the testing apparatus (600).

The testing apparatus (600) is an enclosed container that allows for access into the container via the lid (630). In one or more embodiments, the testing apparatus is leak-proof and sealed to allow for the inside of the container to stay dry from outside moisture. In one or more embodiments, the testing apparatus may have openings to allow oil or any other liquid to drain out of the testing apparatus. The openings may be located in the floor or along the bottom portions of the walls. In one or more embodiments, the testing apparatus may contain a separate chamber that collects oil or liquid run-off For example, the testing apparatus may slope towards the second chamber to allow for oil to drain into the chamber to collect. The separate chamber may be accessed at any time to remove the oil or liquid run-off The testing apparatus (600) may be made out of stainless steel, aluminum, copper-aluminum alloy, non-sparking metal, carbon steel, class 1 div 2 explosion proof material, iron, or any other type of oil-resistant material. For example, the testing apparatus (600) may be made out of a stainless steel rated for a class 1 div 2 hazardous area classification to ensure that the testing apparatus operates safely in conditions containing hazardous vapors, gases, or other flammable substances that could result in an explosion.

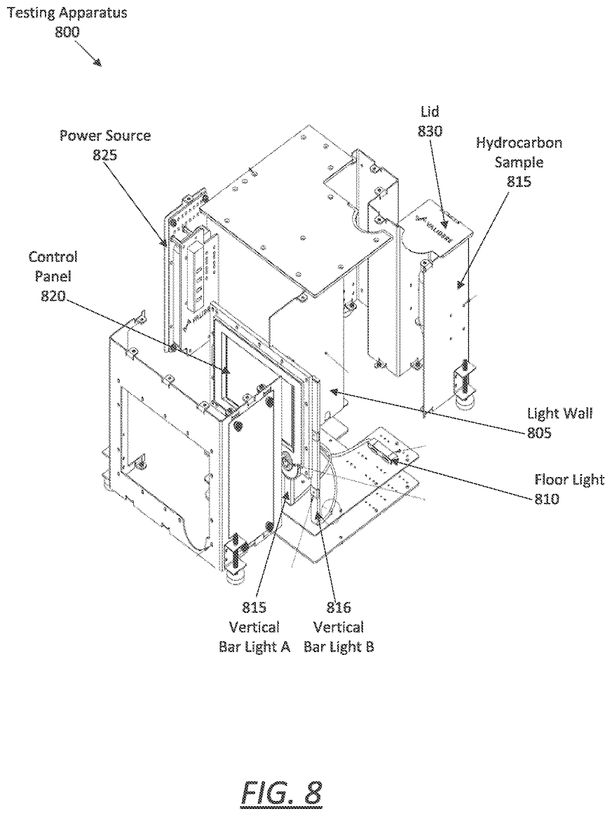

The control panel (610), in accordance with one or more embodiments, is located on the outside portion of the testing apparatus (600). The control panel (610) may be enclosed in a case which then inserts into a side wall. The control panel (600) is discussed in further detail below in FIG. 8.

In one or more embodiments of the invention, the lid (630) is located on the top portion of the testing apparatus (600). The lid (630) may open in a direction towards the user to allow for access inside the testing apparatus (600). In one or more embodiments of the invention, the lid (630) is attached to the testing apparatus (600) via a hinge. In one or more embodiments, the lid (630) is made of the same material as the testing apparatus (600).

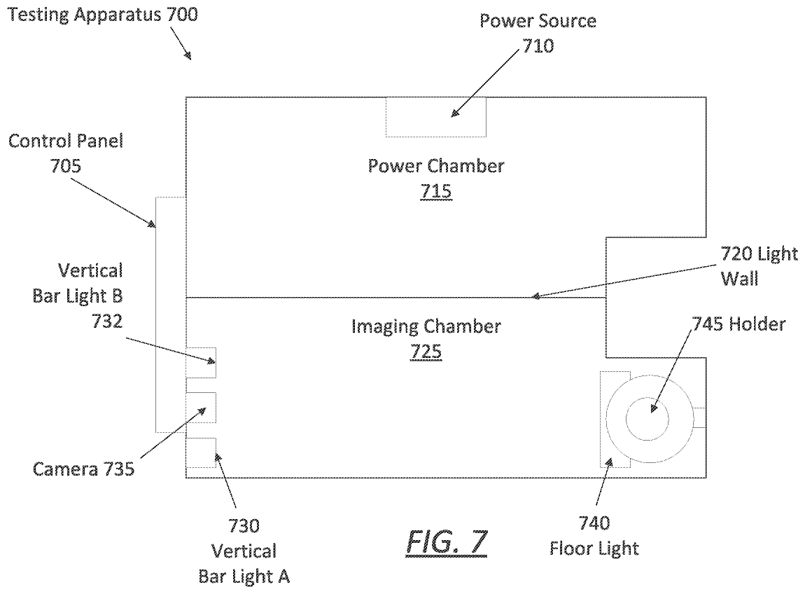

Turning to FIG. 7, a top view of a testing apparatus (700) similar to FIG. 6 is shown. As shown in the figure, the testing apparatus (700) includes of a control panel (705) located on the outside. The inside of the testing apparatus includes of two chambers, a power chamber (715) and an imaging chamber (725). The two chambers are separated by a light wall (720). A power source (710) is located on one wall of the power chamber (715). The imaging chamber (725) includes of a camera (735), vertical light bars (e.g., vertical bar light A (730), vertical bar light B (732), etc.), a floor light (740), and a holder (745).

In one or more embodiments, the control panel (705) is located on a wall on the outer portion of the testing apparatus (700). The control panel (705) may allow a user to access the data and a multitude of images captured by the camera. The control panel (705) is discussed in further detail below and in FIG. 8.

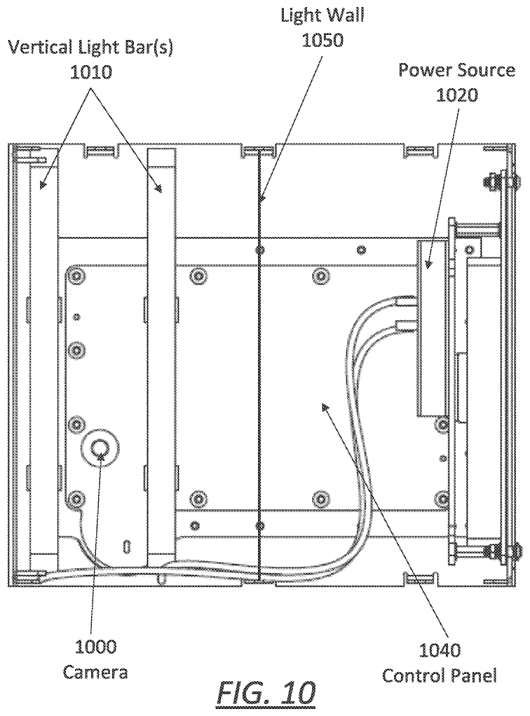

In one or more embodiments, a power source (710) is located in the power chamber (715). The power source (710) may be located on any wall inside the power chamber (715). In one or more embodiments, the power source (710) contains the capacity to store an electric charge or electric battery. In one or more embodiments, the power source (710) receives power from an outside source, such as an electrical outlet or generator, that runs the electronic devices in the testing apparatus (700). The power source (710) is discussed in further detail below and in FIG. 10.

In one or more embodiments, a light wall (720) separates the testing apparatus (700) into multiple chambers. The light wall (720) runs the length of the testing apparatus (700) from one wall to an opposite wall, sufficient to divide the testing apparatus (700) into multiple chambers. In one or more embodiments, the placement of the light wall (720) is dependent upon the light conditions. Prior tests may be done to determine the placement of the light wall (720) to provide the best illumination results for the imaging chamber (725). The light wall (720) may be made of the same material as the testing apparatus (700). In one or more embodiments, the light wall (720) is made of a reflective material to direct the light from the light sources towards a hydrocarbon test sample (not shown) positioned on the holder (745).

A camera (735) is located on a wall inside the imaging chamber (725). In one or more embodiments, the camera (735) is part of the control panel (705) and is controlled by the control panel. For example, a user may view the control panel (705) to focus and adjust the camera (735). The user may then select to capture an image of the hydrocarbon test sample (not shown) by using the control panel (705). In one or more embodiments, the camera (735) is operated separately from the control panel (705). For example, the camera (705) may be a stand-alone device placed in the imaging chamber (725) controlled remotely by a user through a user device such as a mobile device, camera control device, or any other device that may control a camera remotely. In one or more embodiments, the camera (735) may be encased in a material similar to the testing apparatus (700). For example, the camera (735) may be encased in a class 1 div 2 material that allows for the camera to be explosion proof during operation.

In one or more embodiments, the vertical light bars (e.g., vertical bar light A (730), vertical bar light B (732), etc.) are located on the same wall as the camera (735). In one or more embodiments, a vertical light bar (e.g., vertical bar light A (730), vertical bar light B (732), etc.) may be located on either side of the camera (735). The vertical light bars (e.g., vertical bar light A (730), vertical bar light B (732), etc.) are discussed below and in FIGS. 8 and 10.

In one or more embodiments, the floor light (740) is located on the floor of the testing apparatus (700) below the holder (745). In one or more embodiments, the floor light (740) is a LED strip that is the same length as the holder. The floor light (740) is discussed in further detail below and in FIGS. 8 and 10.

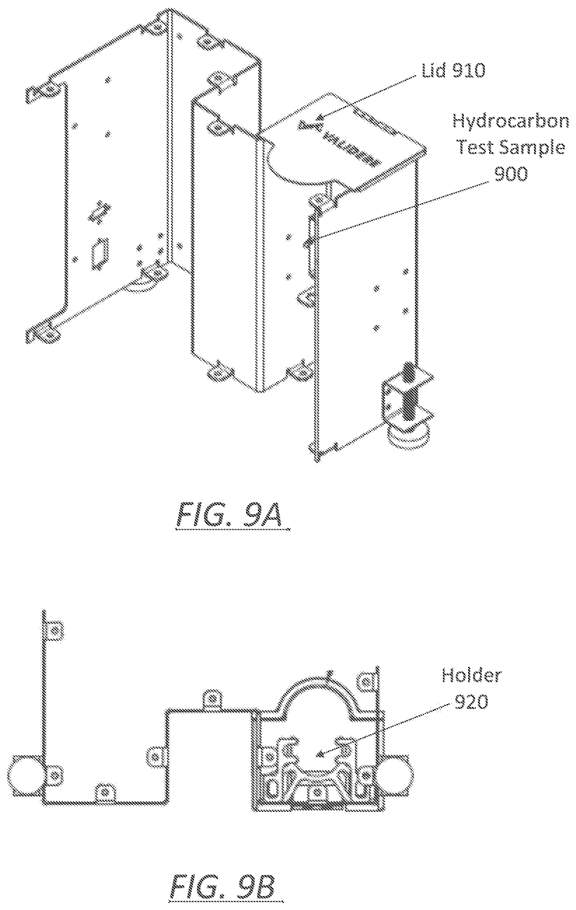

In one or more embodiments, the holder (745) is located on a wall of the testing apparatus (700) opposite of the camera (735). The holder (745) may have a circular design, rectangular design, triangular design, or any other type of design. The holder (745) may be made out of the same material as the hydrocarbon test apparatus (700). In one or more embodiments, the holder (745) is a different material than they testing apparatus (700), such as plastic, stainless steel, carbon steel, iron, or any other type of material. The holder (745) is discussed in further detail below and in FIG. 9.

FIG. 8 shows an exploded view of the testing apparatus (800) from FIG. 6. As shown in FIG. 8, the testing apparatus contains a control panel (820) and a lid (830) on the outside. A light wall (805) divides the testing apparatus (800) in separate chambers. One chamber contains a power source (825) located on a wall of the testing apparatus. The chamber on the opposite side of the light wall (805) contains vertical light bars (e.g., vertical bar light A (815), vertical bar light B (816), etc.) on the inside of the wall sharing the control panel (820). A floor light (810) is located on the floor under the hydrocarbon test sample (815), which is located on a wall opposite of the control panel.

In one or more embodiments, the control panel (820) is located on the outside portion of the testing apparatus (800). As shown in FIG. 8, the control panel (820) may be enclosed in a case which then inserts into a side wall and enclosed by a top and bottom wall. In one or more embodiments, the control panel encasement and the testing apparatus (800) are made of the same material. The material may be stainless steel, aluminum, copper-aluminum alloy, non-sparking metal, carbon steel, class 1 div 2 explosion proof material, iron, or any other type of material.

The control panel (820) may be an input device that allows for user interaction. The control panel may be a tablet computing device, a laptop, a computer, a mobile device, or any other type of computing device that allows for user interaction. In one or more embodiments, the control panel (820) is powered by the power source (825). In one or more embodiments, the control panel (820) houses the camera that captures a multitude of images of the hydrocarbon test sample (815).

As previously discussed, the vertical light bars (e.g., vertical bar light A (815), vertical bar light B (816), etc.) are located on the wall of the testing apparatus (800) housing the control panel (820). In one or more embodiments, a vertical light bar (e.g., vertical bar light A (815), vertical bar light B (816), etc.) is placed on either side of a camera (not shown) inside the testing apparatus (800). The vertical light bars (e.g., vertical bar light A (815), vertical bar light B (816), etc.) may be facing the hydrocarbon test sample (815). In one or more embodiments, the vertical light bars (e.g., vertical bar light A (815), vertical bar light B (816), etc.) is a LED strip.

The floor light (820), in accordance with one or more embodiments, is located on the floor inside of the testing apparatus (800). The floor light (820) may be located underneath the hydrocarbon test sample (815). In one or more embodiments, the floor light (820) is a LED strip that illuminates the hydrocarbon test sample (815).

In one or more embodiments, the hydrocarbon test sample (815) is located underneath a lid (830) on a wall opposite of the control panel (820). The hydrocarbon test sample (815) and the lid (830) are discussed in further detail below and in FIG. 9.

In one or more embodiments, a light wall (805) is a wall located inside the testing apparatus (800) that divides the testing apparatus into two chambers. In one or more embodiments, the power source (825) is located on one wall of the testing apparatus (800) opposite the light wall (805). The power source (825) and the light wall (805) are discussed below and in FIG. 10.

FIGS. 9A and 9B show an isometric and top view, respectively, of one side of the testing apparatus. As shown in FIG. 9A the hydrocarbon test sample (900) is located inside a testing chamber of the testing apparatus. Access to the testing chamber is provided by the lid (910) located on top of the testing apparatus. The hydrocarbon test sample (900) is positioned on a holder (920) attached to the wall of the testing chamber.

In one or more embodiments, the lid (910) is located on the top portion of the testing apparatus, as shown in FIG. 9A. The lid (910) may be attached to the testing apparatus with a hinge to allow the lid to open and close. In one or more embodiments, the lid (910) is attached to the testing apparatus with a sliding rail system that allows for the lid to slide from a closed position to an open position. In one or more embodiments, the lid (910) may be made of the same material as the testing apparatus. The lid may be made out of stainless steel, aluminum, copper-aluminum alloy, non-sparking metal, carbon steel, class 1 div 2 explosion proof material, iron, carbon fiber, plastic, or any other type of material.

In one or more embodiments, the hydrocarbon test sample (900) is located below the lid (910) to allow for access into the testing apparatus. The hydrocarbon test sample (900) may be stored in a container. The container may be a test tube, a cylindrical tube, a flask, a beaker, or any other type of container that holds the hydrocarbon test sample (900). For example, the container may be a tube that is manufactured in accordance with ASTM D4007 or ASTM D0097. The container may be made of glass, plastic, acrylie glass, or any other type of material which allows for the user or control panel to see the hydrocarbon test sample (900).

In one or more embodiments, the holder (920) is located on a wall of the apparatus under the lid (910), as shown in FIG. 9B. The holder (920) may have a circular design to support the hydrocarbon test sample (900), however, the design of the holder (920) is not limited to being circular. The holder may be rectangular, triangle, or any other type of design that supports the hydrocarbon test sample (900). The holder (920) may be made out of the same material as the test apparatus. In one or more embodiments, the holder (920) is a different material than they test apparatus, such as plastic, stainless steel, carbon steel, iron, or any other type of material.

FIG. 10 shows a section view of FIG. 6 taken from the inside of the testing apparatus and facing towards the control panel (1040). As shown in FIG. 10, the control panel (1040) includes of a camera (1000) that faces towards a hydrocarbon test sample (not shown). Vertical light bar(s) (1010) are placed on either side of the camera and are powered by a power source (1020). A light wall (1050) divides the testing apparatus into two chambers.

In one or more embodiments, the camera (1000) is built in to the control panel (1040). The camera (1000) may capture an image and the details of the image, including a time stamp and a geographic location, may all be stored internally on the control panel (1040). For example, the control panel (1040) may be a tablet computing device that includes a camera (1000) on the opposite side of the display screen of the control panel (1040). The control panel may be powered by the power source (1020), which is located in the power chamber. In one or more embodiments, the camera (1000) may be separate from the control panel (1040).

In one or more embodiments, a vertical light bar (1010) is located on both sides of the camera (1000). The vertical light bars (1010) are parallel with respect to one another and run the bottom of the testing apparatus to the top of the testing apparatus. The vertical light bars (1010) may be LED light strips or any other type of light source that illuminates the hydrocarbon testing chamber (not shown). In one or more embodiments, the vertical light bars (1010) may receive power from the power source (1020) via a wire or cable attachment. In one or more embodiments, the vertical light bars (1010) and the power source (1020) are located in separate chambers.

In one or more embodiments, the control panel (1040) is an input device that allows a user to provide input for the testing apparatus. The control panel (1040) may be located on the outside of the testing apparatus to allow the user to have direct access. The control panel (1040) may be a tablet computing device, computer, laptop, mobile device, or any other type of device that allows a user to input and receive data for the testing apparatus. For example, the control panel (1040) may be a tablet computing device attached to the testing apparatus and receives power from the power source (1020).

In one or more embodiments, the power source (1020) is located in the power chamber along a wall. The power source (1020) provides power to the testing apparatus. For example, the power source provides power to the control panel (1040) and the vertical light bar (1010) via a wire, a USB connection, or any other type of connection that transmits power from one source to another. In one or more embodiments, the power source (1020) may be a USB power supply that stores an electrical charge. For example, the power source may be used out in the field or a location that does not have access to a power outlet.

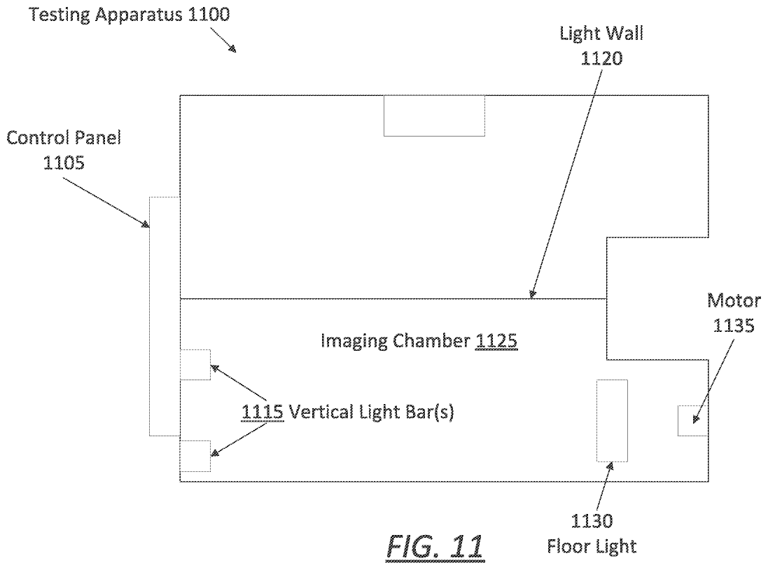

Turning to FIG. 11, an example of a top view of a testing apparatus (1100) is shown in accordance with one or more embodiments. As discussed above, the testing apparatus may include of a set of cameras and a multitude of light sources placed in different locations. As shown in FIG. 11, the testing apparatus (1100) includes of a control panel (1105) located on the outer portion of the testing apparatus. A light wall (1120) divides the testing apparatus (1100) into two separate chambers, an imaging chamber (1125) and a power chamber (not shown). The imaging chamber (1125) includes of vertical light bars (1115) located on the inside of a wall containing the control panel (1105), a floor light (1130) located opposite of the vertical light bars (1115), and a motor (1135).

In one or more embodiments, the vertical light bars (1115) are located on either side of a primary camera (not shown). For example, a vertical light bar (1115) may be on the right side of the camera and a second vertical light bar (1115) may be on the left side of the camera, opposite the first vertical light bar. The two vertical light bars (1115) may be LED lights or any other type of light that extend from the bottom of a wall to the top of the wall. In one or more embodiments, the vertical light bars (1115) are the same height of the camera. In one or more embodiments, the vertical light bars (1115) run along the entire height of the wall. The size of the vertical light bars (1115) should not be limited to these examples, and the size may be determined based on dimensions of the testing apparatus (1100) and the amount of light required to illuminate the imaging chamber (1125). In one or more embodiments, the vertical light bars (1115) may be connected to the camera via a wire, cable, or any other type of connection device. For example, the vertical light bars (1115) may receive an input from the camera via the wire to illuminate the imaging chamber (1125) when the camera captures in image. In one or more embodiments, the vertical light bars (1115) may be connected to an outside source, such as the control panel (1105). For example, the vertical lights (1115) may be programmed by the control panel (1105) to illuminate the hydrocarbon chamber when the camera captures an image.

In one or more embodiments, the floor light (1130) is located beneath the hydrocarbon test sample on the floor of the hydrocarbon test apparatus (1100). The floor light (1130) may be an LED strip or any other type of light source that illuminates the imaging chamber (1125). In one or more embodiments, the floor light (1130) may extend from one wall to an opposite wall. In one or more embodiments, the floor light (1130) may extend the length of the hydrocarbon test sample. The size of the floor light (1130) should not be limited to these examples, and the size may be determined based on dimensions of the testing apparatus (1100) and the amount of light required to illuminate the imaging chamber (1125). In one or more embodiments, the floor light (1130) may be connected to the camera via a wire, cable, or any other type of connection device. For example, the floor light (1130) may receive an input from the camera via the wire to illuminate the imaging chamber (1125) when the camera captures in image. In one or more embodiments, the floor light (1130) may be connected to an outside source, such as the control panel (1105). For example, the floor light (1130) may be programmed by the control panel (1105) to illuminate the hydrocarbon chamber when the camera captures an image.

In one or more embodiments, a motor (1135) is located on a wall adjacent to the hydrocarbon tests sample. The motor (1135) may be battery operated or connected to the power source. The motor (1135) is connected to the hydrocarbon test sample in a manner that will allow the motor to rotate the hydrocarbon test sample. For example, the motor (1135) may contain a belt which is joined between a rotating shaft on the motor and the hydrocarbon test sample. The rotation of the motor shaft will allow the turn the belt which will allow the hydrocarbon test sample to rotate.

In one or more embodiments, the motor (1135) may be located on a wall adjacent to the camera (not shown). The motor (1135) is connected to the camera in a manner that will allow the motor to rotate the camera. For example, the motor (1135) may contain a belt which is joined between a rotating shaft on the motor and the camera. The rotation of the motor shaft will allow the turn the belt which will allow the camera to rotate.

In one or more embodiments, the motor (1135) may be located on a wall adjacent to the vertical light bar (1115). The motor (1135) is connected to the vertical light bar (1115) in a manner that will allow the motor to rotate the vertical light bar (1115). For example, the motor (1135) may contain a belt which is joined between a rotating shaft on the motor and the vertical light bar (1115). The rotation of the motor shaft will allow the turn the belt which will allow the vertical light bar (1115) to rotate.

In one or more embodiments, the testing apparatus (1100) is divided in two separate chambers by a light wall (1120). The light wall (1120) runs the length of the testing apparatus (1100) from one wall to the opposite wall. For example, the light wall (1120) may be placed on the center point of the wall which contains the vertical light bars (1115) and run perpendicular from the wall, across the imaging chamber (1125), to the opposite wall. In one or more embodiments, the placement of the light wall (1120) is dependent upon the light conditions. Prior tests may be done to determine the placement of the light wall (1120) to provide the best illumination results for the imaging chamber (1125).

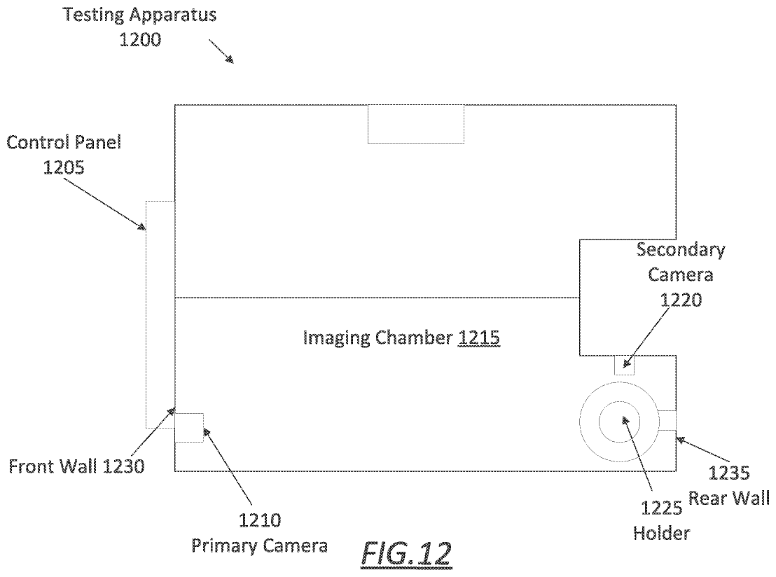

FIG. 12 shows an example of a top view of a testing apparatus (1200) in accordance with one or more embodiments. As discussed above, the testing apparatus (1200) may include of a set of cameras and a multitude of light sources placed in different locations. As shown in FIG. 12, the testing apparatus (1200) includes of a control panel (1205) and a imaging chamber (1215), which includes of a primary camera (1210) located on a front wall (1230) opposite of a hydrocarbon test sample (1240), which is positioned on a holder (1225), located on a rear wall (1235). A secondary camera (1220) is located on a wall perpendicular to the primary camera.

In one or more embodiments, the primary camera (1210) and the secondary camera (1220) capture a multitude of images of the hydrocarbon test sample (1240) and send the multitude of images to the control panel (1205) for analyzing. In one or more embodiments, the primary camera (1210) and secondary camera (1220) capture a multitude of images in unison. For example, the primary camera (1210) and secondary camera (1220) capture a multitude of images under the same lighting conditions at the same time. This allows for the multitude of images of the hydrocarbon test sample (1240) to be captured from multiple angles to provide greater accuracy in the analysis.

In one or more embodiments, the primary camera (1210) and secondary camera (1220) capture a multitude of images under different lighting conditions. For example, the primary camera (1210) may capture a multitude of images while the hydrocarbon test sample (1240) is illuminated by a UV light source and a multitude of other light sources. The secondary camera (1220) may then capture a multitude of images while the hydrocarbon test sample (1240) is illuminated by only the multitude of other light sources.

In one or more embodiments, the multitude of images captured by the primary camera (1210) and secondary camera (1220) are time stamped and tagged with a geographic location. For example, the primary camera (1210) and secondary camera (1220) may upload the multitude of images instantaneously to the control panel (1205) and the control panel assigns a time stamp and geographic location to the multitude of images using internal GPS. In one or more embodiments, the primary camera (1210) and secondary camera (1220) assign the time stamp and geographic location to the multitude of images before the multitude of images are uploaded to the control panel (1205).

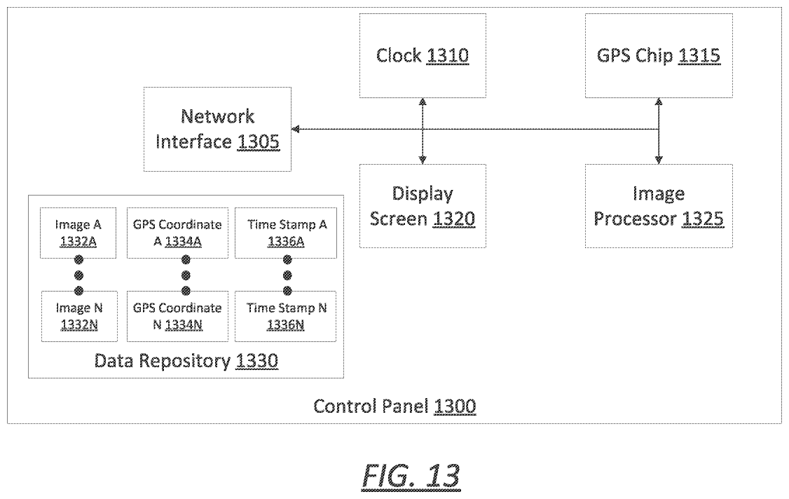

FIG. 13 shows a system in accordance with one or more embodiments. In one or more embodiments, the system is contained within a control panel (1300) which contains a network interface (1305), a data repository (1330), a display screen (1320), an image processor (1325), a clock (1310), and a Global Positioning System (GPS) chip (1315). Each of these components is described below.

As previously discussed, the control panel (1300) may be any type of input device (e.g., a computing tablet, a laptop, a mobile device, a computer). The control panel (1300) contains a data repository (1330) for storage. In one or more embodiments, the data repository (1330) is any type of storage unit and/or device (e.g., a file system, database, collection of tables, or any other storage mechanism) for storing data/information. Specifically, the data repository (1330) may include hardware and/or software. Further, the data repository (1330) may include multiple different storage units and/or devices. The multiple different storage units and/or devices may or may not be of the same type or located at the same physical site. Further, the data repository (1330) includes functionality to store, at least, multiple images (e.g., image A (1332A), image N (1332N)).

In one or more embodiments, multiple images (e.g., image A (1332A), image N (1332N)) are stored in the data repository (1330). Image A (1332A) may be an image captured of the hydrocarbon test sample using three light sources, while image N (1332N) may be an image of the hydrocarbon test sample captured using one light source and a UV light source.

In one or more embodiments, the data repository (1330) stores GPS coordinates (e.g., GPS coordinate A (1334A), GPS coordinate N (1334N)). The GPS coordinates may contain information related to the location of an image when the image is captured. The GPS coordinates may be assigned to an image when the image is captured. For example, image A (1332A) may be captured in a location and GPS coordinate A (1334A) contains the data associated with the location. GPS coordinate A (1334A) is then assigned to image A (1332A). Image N (1332N) may be captured in a second location and GPS coordinate N (1334N) contains the data associated with the second location. GPS coordinate N (1334N) is then assigned to image N (1332N).

In one or more embodiments, the data repository (1330) stores time stamps (e.g., time stamp A (1336A), time stamp N (1636N). The time stamps contain data related to the time that an image was captured. For example, image A (1332A) may be captured at 6:36 pm. Time stamp A (1336A) is then associated with 6:36 pm and image A (1332A). Image N (1332N) may be captured at 6:38 pm. Time stamp N (1636N) is then associated with 6:38 pm and image N (1332N).

Returning to the control panel (1300), the control panel contains a network interface (1305) to send and receive data. The network interface (1305) may be a local area network (LAN), a wide area network (WAN) such as the Internet, mobile network, Bluetooth, or any other type of network The network interface (1305) may have capability to send and receive images to and from the control panel (1300) to be stored on a data repository (1330). For example, image A (1332A) may be captured by a camera and received by the control panel (1300) through Bluetooth using the network interface (1305). In one or more embodiments, the control panel (1300) may try to send image A (1332A) to a separate computing device without the network interface (1305) detecting a useable network Image A (1332A) will remain in the data repository (1330) until the network interface (1305) detects a network to send image A (1332A) to the separate computing device.

The control panel (1300) may contain an image processor (1325). The image processor (1325) is configured to perform instructions on the control panel (1300) (e.g., image processing, algorithms). The image processor(s) (1325) may be an integrated circuit for processing instructions. For example, the image processor(s) may be one or more cores, or micro-cores of a processor.

The control panel (1300) may contain a display screen (1320) to display content to the user. The display screen may be a liquid crystal display (LCD), a plasma display, touchscreen, cathode ray tube (CRT) monitor, projector, or other display device. For example, the display screen (1320) may display image A (1332A) to the user. In one or more embodiments, the user takes actions based on being presented image A (1332A). The user may choose to send image A (1332A) to a separate computing device via the network face (1305), choose to store image A (1332A) in the data repository (1330), or choose to run image processing based on the instructions in the image processor (1325).

In one or more embodiments, the control panel (1300) may contain a clock (1310). The clock (1310) may display the time to the user and track time internally on the control panel (1300). For example, the clock (1310) may provide time stamp A (1336A) to image A (1332A) based on the time that image A was captured by the control panel (1300).

In one or more embodiments, the control panel (1300) may contain a GPS chip (1315). The GPS chip (1315) has capability to track the location of the control panel (1300) and to store the information in the data repository (1330). For example, the GPS chip (1315) may provide the location of image A (1332A) in the form of GPS coordinate A (1334A) based on the location that image A (1332A) was captured by the control panel (1300).

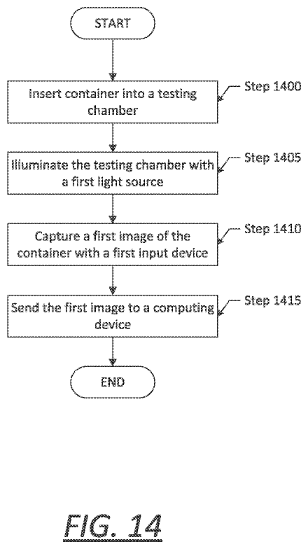

Turning to FIG. 14, a flow chart describing a method for capturing an image of a container is shown. In step, 1400, a container is inserted into a test chamber. The testing chamber may contain a lid that opens to allow the container to be inserted. In one or more embodiments, the testing chamber may rotate to reveal an opening for the container to be inserted.

In step 1405, the testing chamber is illuminated by a first light source. In one or more embodiments, the first light source is directed towards the container in the testing chamber. In one or more embodiments, the first light source is directed towards a reflective wall that allows for the light to reflect off the wall onto the container. In one or more embodiments, one or more light sources may be used to illuminate the testing chamber.

In step 1410, a first image of the container is captured by a first input device. The first input device is located in a manner that allows for the image contents to contain the entire container. In one or more embodiments, a second input device captures an image of the container. In one or more embodiments, multiple images of the container are captured.

In step 1415, the first image is sent to a computing device from the first input device. The first image is initially stored on the first input device. The first image is then sent to the computing device over a network (e.g., Bluetooth, a LAN connection network, WiFi). In one or more embodiments, the computing device is a control panel connected to the testing apparatus. In one or more embodiments, the computing device is external from the testing apparatus.

Thus, the embodiments and examples set forth herein were presented in order to best explain various embodiments and their particular application(s) and to thereby enable those skilled in the art to make and use the embodiments. However, those skilled in the art will recognize that the foregoing description and examples have been presented for the purposes of illustration and example only. The description as set forth is not intended to be exhaustive or to be limiting to the precise form disclosed.

While many embodiments have been described, those skilled in the art, having benefit of this disclosure, will appreciate that other embodiments can be devised which do not depart from the scope.

What is claimed is:

1. A testing apparatus, comprising:
a chamber, comprising:
a holder configured to position a container;
a first input device and a second input device, each partially outside the chamber and configured to:
generate a first image of the container, and
generate a second image of the container;
a first light source configured to illuminate the container for the first image; and
a second light source configured to illuminate the container for the second image.

2. The testing apparatus of claim 1, further comprising:
a control panel configured to operate at least the first input device.

3. The testing apparatus of claim 2, wherein the first input device is a primary camera communicatively coupled to the control panel via a wireless network.

4. The testing apparatus of claim 1, wherein the first input device is a primary camera located on the front wall and the secondary input device is a secondary camera located on the rear wall.

5. The testing apparatus of claim 4, further comprising:
a first motor connected to the container, wherein the first motor rotates the container;
a second motor connected to the first input device, wherein the second motor rotates the first input device; and
a third motor connected to the first light source, wherein the third motor rotates the first light source.

6. The testing apparatus of claim 1, wherein the primary camera is attached to an input device.

7. The testing apparatus of claim 1, wherein the first light source is located on a top wall of the chamber and the second light source is located on a bottom wall of the chamber.

8. The testing apparatus of claim 1, wherein the second light source is an ultraviolet (UV) light source.

9. The testing apparatus of claim 1, further comprising:
a plurality of horizontally-aligned light sources in the chamber, wherein
the first light source is aligned vertically in the chamber and the second light source is aligned vertically opposite the first light source.

10. The testing apparatus of claim 1, wherein the chamber is cylindrical.

11. The testing apparatus of claim 10, wherein the first light source is located on a top wall of the chamber and the second light source is located on a bottom wall.

12. The testing apparatus of claim 1, wherein the chamber is capable of withstanding extreme conditions.

13. A method for testing a hydrocarbon test sample, comprising:
obtaining a first image of the hydrocarbon test sample via a first input device, wherein the first input device is a primary camera configured to capture the first image while a plurality of light sources illuminates the hydrocarbon test sample;
sending the first image from the first input device to a control panel, wherein the control panel labels a plurality of layers on the first image; and
determining a water cut of the hydrocarbon test sample based on labeling of plurality of layers of the first image.

14. A method for hydrocarbon testing comprising:
inserting a container into a testing chamber;
illuminating the testing chamber using a first light source;
capturing a first image of the container with a first input device in proximity to the first light source illuminating the testing chamber; and
sending the first image of the container from the input device to a computing device.

15. The method of claim 14 comprising:
illuminating the testing chamber using a second light source, wherein the second light source is an ultraviolet (UV) light source; and
capturing a second image of the container in proximity to the second light source illuminating the testing chamber.

16. The method of claim 14, further comprising:
rotating the container, via a motor; and
capturing a third image of the container in proximity to the first light source illuminating the testing chamber.

17. The method of claim 14, further comprising:
a second input device configured to capture a second image of the container, wherein the second input device is perpendicular to the first input device.

18. The method of claim 14, further comprising:
rotating the first input device, via a motor; and
capturing a fourth image of the container in proximity to the first light source illuminating the testing chamber.

19. A testing apparatus, comprising:
a chamber, comprising:
a holder configured to position a container;
a first input device, partially outside the chamber and configured to:
generate a first image of the container; and
a first light source configured to illuminate the container for the first image.

20. The testing apparatus of claim 19, wherein the first light source is an ultraviolet (UV) light source.
